(19) United States
(12) Patent Application Publication
(10) Pub. No.: US 2002/0137114 A1
(43) Pub. Date: Sep. 26, 2002

(54) Method of detecting PrP protein and kits therefor

(76) Inventors: Dirk Voelkel, Vienna (AT); Klaus Zimmermann, Vienna (AT); Peter Turecek, Klosterneuburg (AT); Hans-Peter Schwarz, Vienna (AT)

Correspondence Address: Baxter Healthcare Corporation, P.O. Box 15210, Irvine, CA, 92614, US

(21) Appl. No.: 10/051,413

(22) Filed: Jan. 18, 2002

Related U.S. Application Data
(60) Provisional application No. 60/263,022, filed on Jan. 19, 2001.

(51) Int. Cl. G01N 33/53; G01N 33/537; G01N 33/543

(52) U.S. Cl. 435/7.92

(57) ABSTRACT

The invention relates to a method for the detection of neurological disorders in a patient comprising (a) measuring the concentration of PrP protein in a biological fluid sample of said patient; and (b) determining whether said concentration of said PrP protein is above or below a predetermined threshold value, whereby the concentration above said predetermined threshold value identifies a patient with a neurological disorder, a method for the detection and quantification of PrP protein and pathogenic PrP^{res} protein in a sample, and a kit comprising a set of reagents to determine the concentration of PrP protein and pathogenic PrP^{res} protein in a sample.

### CROSS REFERENCE TO RELATED APPLICATIONS

This application claims priority to U.S. Provisional Application No. 60/263,022, filed Jan. 19, 2001, which application is incorporated by reference in its entirety.

### FIELD OF THE INVENTION

The present invention is directed to a method for the detection of neurological disorders in a patient as well as a method for measuring the concentration of PrP protein in a sample.

### BACKGROUND OF THE INVENTION

Transmissible spongiform encephalopathy (TSE) represents a spectrum of diseases affecting both humans and animals. Examples of TSE are scrapie in sheep and goats or bovine spongiform encephalopathy (BSE) in cattle. In humans, TSE is known as Creutzfeldt-Jakob disease (CJD). The most common form, sporadic CJD (sCJD), is a rare progressive and fatal neurodegenerative disorder with a worldwide incidence of 0.5-2 new cases per year and per million population. CJD is characterized clinically by dementia, ataxia and myoclonus, and histopathologically by astrogliosis, dendritic spongiosis and neuronal loss. In addition to sCJD and hereditary (familial) forms, acquired forms of CJD also exist, for example, Kuru resulting from the consumption of contaminated material, or iatrogenic CJD acquired through transplantations and medical procedures.

As early as 1967, Griffith postulated a self-replicating infectious agent as the cause of scrapie, and in 1982, Prusiner proposed the “prion hypothesis”, which assumes that the prion protein, a small proteinaceous particle not associated with nucleic acids, is the major and possibly only infectious particle in such diseases. The prion protein is mainly a phosphatidylinositol glycolipid-anchored cell surface protein¹ and is predominantly found on neurons², glia³, B-lymphocytes,^{4,5} in peripheral blood mononuclear cells⁶ and on platelets.⁷ Whole blood and plasma have been reported to contain soluble PrP that is probably actively released from platelets.⁸ The cellular function of the prion protein is unclear. Its capability for binding bivalent metals such as copper and its superoxide dismutase activity have prompted speculation that it is directly linked to cellular resistance to oxidative stress and thus important for synaptic activity. Experiments with PrP-knockout mice have suggested that PrP might play a role in the regulation of sleep/activity rhythms. Overexpression of PrP in transgenic mice induced a spongiform-like degeneration of the nervous system, and synthesized fragments of PrP possess neurotoxic properties.

The infectivity of prion diseases is thought to be caused by a structural change of the prion protein from the soluble form normally found in cells (PrP^{sen}—sensitive to proteinase K treatment) into an insoluble form (Prp^{res}—partially resistant to proteinase K) that has a tendency to form fibrils. A new variant of CJD (vCJD) was observed in humans approximately ten years after the 1986 outbreak of BSE in the United Kingdom. In contrast to patients with sCJD, those with vCJD showed pathogenic PrP^{res} fibrils in lymphatic organs such as the tonsils. The appearance of vCJD led to concern regarding the transmission of animal forms of TSE to humans. Evidence supporting the hypothesis that BSE had been transmitted to humans was provided by experiments with transgenic mice expressing bovine prion protein and by Western blot analysis of the glycoform ratio of PrP^{res}. Furthermore, experiments in which mice were inoculated with brain extracts, either from patients with vCJD or sCJD or from cattle affected by BSE, demonstrated that the “signature” of disease was identical in both BSE and vCJD.

These findings led to new uncertainties and intense discussions regarding the safety of plasma products. Although transmission of TSE through whole blood has been demonstrated in animals, the amount of infectious agent needed to transmit disease by an intravenous route makes transmission by therapeutic blood products unlikely, and no case of transmission of CJD by blood transfusion or administration of blood products in humans has ever been identified. However, continued concern regarding these issues has resulted in increased demand for a rapid assay capable of detecting infectious prions in blood, see for example WO 99 63 109.

Up to the present, a definitive diagnosis of vCJD has only been possible by post mortem immunohistochemical identification of the pathological PrP^{res} in brain tissue or in tonsils from affected patients. The most important method for diagnosis on the basis of blood samples has thus far been the detection of the resistant PrP^{res} after proteinase K treatment and Western blotting analysis. However, due to its lack of sensitivity, this method is unsuitable for routine screening of blood or plasma. Several proteins such as S-100 proteins,⁹ tau proteins,¹⁰ neuron specific enolase,¹¹ creatine kinase, myelin basic protein and 14-3-3 proteins^{12,13,14} have been discussed as surrogate markers for CJD. These proteins are however all problematic for screening assays (especially for CJD) in blood and plasma samples; for example, detection levels may be very low, some do not exist in the cerebrospinal fluid, some cross-react with non-brain isoforms, or levels may change in the late stages of disease.^{15,16}

It is therefore necessary to have a simple and rapid method which enables the detection of even very low concentrations of total PrP proteins as well as separately PrP^{sen} and pathogenic PrP^{res} proteins in a sample which method can be used for rapid screening assays.

Other neurological disorders, especially in the early stages are often difficult to detect and diagnose. Some neurological diseases vary in the symptoms so that often a plurality of methods for diagnosis are necessary to even diagnose a neurological disorder in general. Such methods are time consuming for the doctors and laboratory assistants as well as for the patients which is according to the state of illness of the patient very tiring and difficult for all who are concerned. Furthermore, these tests require highly technical and expensive apparatus so that these tests cannot be carried out in any laboratory but only in very specialized laboratories and hospitals.

A rapid diagnostic assay which can be used for routine screening for the detection of neurological disorders is therefore one object of the present invention.

Another object of the present invention is a highly sensitive method to detect PrP proteins in a sample.

A further aspect of the present invention is a kit to determine the concentration of PrP protein in a sample.

### SUMMARY OF THE INVENTION

In accordance with an aspect of the invention a method for the detection of neurological disorders in a patient is provided comprising

(a) measuring the concentration of PrP protein in a biological fluid sample of said patient; and

(b) determining whether said concentration of said PrP protein is above or below a predetermined threshold value, whereby the concentration above said predetermined threshold value identifies a patient with a neurological disorder.

The measuring of the PrP protein is preferably carried out immunologically. In this case the biological fluid sample can be incubated with a labelled anti-PrP ligand after which the amount of bound labelled ligand is determined. For the incubation, the PrP protein which is present in said biological fluid sample can be immobilized to a solid phase and labelled anti-PrP ligand can be added.

The immobilization may comprise immobilizing a first anti-PrP ligand to said solid phase and adding said biological fluid sample so that said PrP protein binds to said first anti-PrP ligand and is immobilized to said solid phase.

The determination of bound labelled ligand may comprise

(a) adding a substrate which reacts with the label of said ligand;

(b) the reaction between said substrate and said label resulting in a quantifiable signal; and

(c) measuring said signal.

Preferably the method for determining the amount of bound labelled ligand further comprises comparing said signal of said biological fluid sample with a measured signal of a control sample comprising a defined concentration of PrP protein.

The quantifiable signal is preferably a quantifiable optical signal whereby the reaction between said substrate and said label may result in a fluorescent signal. In the case the reaction results in the formation of a fluorescent chelate the substrate is selected from the group consisting of europium, terbium, gadolinium, samarium and dysprosium. Furthermore, the substrate may have bound thereto avidin or streptavidin. Preferably, the labelled ligand is biotinylated.

According to a preferred embodiment of the present invention the neurological disorders are selected from the group consisting of Creutzfeldt-Jakob disease (CJD), Alzheimer disease, depression by dementia of Alzheimer, Parkinson disease, dementia, inflammatory brain damage, Alcoholism, or state of confusion.

A second aspect of the disclosed invention relates to a method for the detection and quantification of PrP protein in a sample comprising

(a) immobilizing a first anti-PrP ligand to a solid phase;

(b) incubating said sample with said first anti-PrP ligand so that said PrP protein binds to said first ligand;

(c) adding a second labelled anti-PrP ligand so that said second ligand binds to said PrP protein;

(d) adding a substrate which reacts with the label of said ligand;

(e) the reaction between said substrate and said label resulting in a quantifiable signal;

(f) measuring said signal; and

(g) determining the concentration of PrP protein in said sample, preferably by comparing said signal of said sample with a signal of a control sample comprising a defined concentration of PrP protein.

A further embodiment of the present invention concerns a method for the detection and quantification of pathogenic PrP^{res} protein in a sample whereby before immobilizing a first anti-PrP ligand to a solid phase PrP^{sen} protein in said sample is eliminated by incubating said sample with proteinase K after which the proteinase K digested sample is incubated with said first anti-PrP ligand so that non-digested PrP^{res} protein binds to said first ligand. The following steps are carried out as mentioned above.

Another aspect of the present invention is a kit comprising a set of reagents to determine the concentration of PrP protein in a sample, said set of reagents comprising

(a) a solid phase having bound thereto a first anti-PrP ligand;

(b) a first reagent comprising a second labelled anti-PrP ligand;

(c) a second reagent comprising a substrate which reacts with the label of said ligand, the reaction between said substrate and said label resulting in a quantifiable signal; and

(d) optionally a third reagent comprising a control sample comprising a defined concentration of a PrP protein.

Preferably, the quantifiable signal is a quantifiable optical signal. Said substrate may e.g. be selected from the group consisting of europium, terbium, gadolinium, samarium, and dysprosium. The substrate can have bound thereto avidin or streptavidin; the labelled ligand can be biotinylated; the sample can be a plasma sample of said patient.

According to a further aspect, the kit comprises a set of reagents to determine the concentration of pathogenic PrP^{res} protein in a sample, said set of reagents further comprising a further reagent comprising proteinase K and optionally a fifth reagent comprising a control sample with a defined concentration of a PrP^{res} protein.

Definitions

“Anti-PrP ligand” refers to a monoclonal or polyclonal antibody, a peptide, phage, protein, DNA or RNA or other non-biological polymers, which specifically recognizes all PrP protein isoforms, e.g. pathogenic PrP^{res} proteins as well as normal PrP^{sen} proteins.

“Biotinylated” refers to a biotin moiety covalently attached to a protein or peptide for the purpose of reacting e.g. with avidin or streptavidin in a detection assay.

“Immobilizing” in the context of the proteins or peptides refers to the binding or attaching of the protein/peptide to solid supports by conventional means.

“Label” refers to any indicator substance which can be precisely quantified and therefore give information on the amount of bound ligand, in particular antibody. The label may either allow direct quantification (in which case the label may be e.g. an isotope, fluorophore, or enzyme, etc.) or indirect quantification (the label may be e.g. biotin or digoxigenin which will be detected by a secondary reagent).

“Measuring” comprises any method known to the person skilled in the art which enables to quantify the concentration of PrP protein in a sample. This may comprise chemical, microbiological, physical techniques, etc.

“Biological fluid” comprises any body liquid such as for example blood, plasma, plasma fraction or cerebrinal fluid.

“Neurological disorder” relates to any disorder of the nervous system or any disorder being mainly related to or having a main impact on the nervous system.

“Patient” relates to any human being whether or not this person is affected by a neurological disorder on whom the method according to the present invention is carried out. The person may or may not show symptoms of a neurological disorder. Therefore, this method can be carried out on patients who are affected by a neurological disorder, who are suspected to have a neurological disorder or simply on a healthy person for e.g. statistical reasons.

“PrP protein” if it is not specified relates to any isoform of the prion protein. It therefore comprises the soluble form normally found in cells (cellular PrP^{sen}—sensitive to proteinase K treatment) as well as the insoluble form (PrP^{res}—partially resistant to proteinase K treatment).

“Solid support” refers to any insoluble material which can provide a substrate upon which to immobilize ligands. Such substrates may include nylon, amino or carboxy activated plastics, glass, cellulose and the like.

“Substrate” relates to any substance which specifically reacts with the label, e.g. an enzyme, a chemical substance, etc. In order to amplify the resulting signal the anti-PrP ligand may have connected thereto a plurality of labels and/or the label may be modified so a plurality of substrate molecules may bind to one label.

“Threshold value” relates to a concentration value which will generally be the median plasma concentration of PrP in healthy plasma donors. It is possible to take the known general median plasma concentration in healthy plasma donors according to the literature, however it is advisable to determine the plasma concentration of PrP in healthy donors parallel to the patients and according to the same method as the determination of the PrP plasma concentration in the patient. This allows a direct and very precise comparison since the threshold value may vary according to the sensitivity of the detection method. The threshold value may also be defined by concentration values determined in healthy (normal) samples taken earlier (in a healthy state) from the same person. Examples for such threshold values may be for example 2 ng/ml and 10 ng/ml, preferably 6.0 ng/ml and 6.5 ng/ml.

### BRIEF DESCRIPTION OF THE DRAWINGS

FIG. 1 shows the scheme of the sandwich ELISA for testing of cellular PrP^{sen} and pathogenic PrP^{res} in human plasma samples.

FIG. 2 shows the calibration of the sandwich ELISA with recombinant hamster PrP.

FIG. 3 shows the stability of PrP^{sen} in plasma samples.

FIG. 4 shows the result of spiking of plasma samples.

FIG. 5 shows a Western blot of proteinase K resistant PrP^{res}.

FIG. 6 shows the comparison of the PrP^{sen} concentration in plasma samples from healthy plasma donors, patients with CJD, and patients with other neurological diseases.

FIG. 7 shows the correlation between age and PrP^{sen} concentration in plasma samples from healthy plasma donors and patients with definite or probable CJD.

### DETAILED DESCRIPTION

It has been surprisingly found that the concentration of PrP protein in a plasma sample of a patient with a neurological disorder is higher than the concentration of PrP protein in a plasma sample of a healthy patient. Therefore, the method according to the present invention in which the concentration of PrP in a plasma is measured and then compared to a predetermined threshold value provides a simple and rapid method for diagnosing neurological disorders in a patient. Since the method is carried out on a plasma sample of a patient it is not necessary to analyze further organs, e.g. brain, tissues, etc. in order to detect that the patient suffers from a neurological disorder. It is only necessary to take a blood sample from the patient which can be carried out quickly and without requiring time and active help by the patient. Therefore, for the present method it is unimportant in what state the patient is to the contrary of known methods where active help of the patient, e.g. in psychological and motoric tests, is needed.

Due to the rapidity and simplicity of the present method it is possible to screen a big number of patients for neurological disorders. The method according to the present invention is therefore suitable for routine analyses in a hospital or clinic as well as for analyses with respect to hereditary neurological disorders in a specific people or family.

The present method is furthermore cost saving and is therefore suitable also for statistical analyses in any part of the world.

Preferably, the measuring of the PrP protein is carried out immunologically. Any immunological method known to the person skilled in the art may be applied. However, it is necessary that at least one anti-PrP ligand is used which will bind to the PrP protein. Immunological methods are highly precise and sensitive methods for detecting molecules and are therefore particularly advantageous for the measuring of the PrP protein.

In one preferred embodiment the biological fluid sample is incubated with a labelled anti-PrP ligand after which the amount of bound labelled ligand is determined. Any already known and used or newly produced, monoclonal or polyclonal, anti PrP antibody, phage, DNA or RNA, peptide, protein or non-biological polymer may be used.

Labelling can be achieved by one of the many different methods known to those skilled in the art. In general, labelling and detection of bound antibodies may be divided into general types: direct and indirect. Direct methods employ either covalent attachment or direct enzymatic incorporation of the signal generating moiety (e.g., isotope, fluorophore, or enzyme) to the ligand. Indirect labelling uses a hapten (e.g., biotin or digoxigenin) bound to the ligand, followed by detection of the hapten with a secondary reagent such as streptavidin or ligand conjugated to a signal generating moiety (e.g. fluorophore or signal generating enzymes such as alkaline phosphatase or horseradish peroxidase).

For example, methods of determining the amount of bound labelled ligand include, without limitation, fluorescent, radioisotope, chemiluminescence, bioluminescence, colorimetric and electrochemiluminescence labeling. Many known labeling techniques require a wash step to remove excess target from the antigen/ligand solution.

Fluorescent labeling is suitable for this invention for several reasons. The fluorescent labeling procedures are simpler than chemiluminescent methods since the latter requires enzymatic reactions and detection in the solution state, and the fluorescent labeling approach can be modified to achieve the highest signal to noise ratio among the safest labeling techniques by utilizing secondary linker chemistries that enable the attachment of hundreds of fluorescent dye molecules per target molecule.

The typical fluorescent dyes to be considered include commercially available agents such as ethidium bromide, as well as the novel dyes proposed in the affiliated chemistry component. These labeling agents have intense absorption bands in the near UV (300-350 nm) range while their principle emission band is in the visible (500-650 nm) range of the spectrum.

The PrP protein which is present in said plasma sample may be immobilized to a solid phase and the labelled anti-PrP ligand is added to the bound PrP protein. Non limitative examples for the solid phase are microtiter plates, tubes, beads of polystyrole, polyphenyl chloride, etc. The immobilization may be adsorption, covalent binding, etc.

Immobilizing the PrP protein to the solid phase may comprise immobilizing a first anti PrP ligand to a solid phase and adding the plasma sample so that the PrP protein binds to the first anti-PrP ligand and is immobilized to the solid phase.

In one preferred form the determination of bound labelled ligand is carried out by adding a substrate which reacts with the label. The reaction between the substrate and the label results in a quantifiable signal, which signal is measured wherein optionally the signal of the plasma sample is compared with a measured signal of a control sample comprising a defined concentration of PrP protein.

In the case that the method according to the present invention is calibrated the measured signal can be directly interpreted, and the concentration of PrP protein in the sample can be directly determined. However, for a precise and correct interpretation of the measured signal a control sample comprising a defined concentration of PrP protein may be tested parallel to the plasma sample in which case the signal from the plasma sample would be compared to the signal of the control sample and precisely interpreted. However, this method is not limited to only one parallel control sample, the interpretation of the measured signal of the plasma sample is more precise and easier if a row of different dilutions of the control sample are tested.

Preferably the reaction between the substrate and the label results in a fluorescent signal. The reaction may also result in a formation of a fluorescent chelate, preferably wherein a metal ion is stably retained, e.g. a lanthanide metal ion. As an alternative to ethidium-based fluorescent reporter groups, which are known for their tendency to absorb nonspecifically to surfaces causing increased signal background, the use of aromatic lanthanide (Ln) chelators may be used in the instant invention. Although the lantanoid ions (Tb and Eu specifically) have luminescent yields near to 1, and emission lifetime years to 100, they absorb light weakly and are therefore poor luminescent dyes. However, when chelated by an appropriately chosen aromatic donor, energy transfer can occur resulting in high overall luminescent yields. DPA (dipiccolimic acid) is the prototype for such an aromatic Ln chelator, and has excellent photophysical properties.

Preferably the substrate is selected from the group consisting of europium, terbium, gadolinium, samarium and dysprosium.

These substrates may have bound thereto avidin or streptavidin. In case the substrate is avidin or streptavidin the labelled ligand can be biotinylated. A biotin-avidin or biotin-streptavidin association will be formed therefrom. This is a simple and efficient way of creating a substrate and a label which will specifically react with one another.

The neurological disorders may be selected from the group consisting of Creutzfeldt-Jakob disease (CJD), Alzheimer disease, depression by dementia of Alzheimer, Parkinson disease, dementia, inflammatory brain damage, alcoholism, or state of confusion.

A further aspect of the present invention is a method for the detection and quantification of PrP protein in a sample comprising

(a) immobilizing a first anti-PrP ligand to a solid phase;

(b) incubating said sample with said first anti-PrP ligand so that said PrP protein binds to said first ligand;

(c) adding a second labelled anti-PrP ligand so that said second ligand binds to said anti-PrP protein;

(d) adding a substrate which reacts with the label of said second bound ligand;

(e) the reaction between said substrate and said label resulting in a quantifiable signal;

(f) measuring said signal; and

(g) determining the concentration of PrP protein in said sample, preferably by comparing said signal of said sample with a signal of a control sample comprising a defined concentration of PrP protein.

To the contrary of the method described by MacGregor et al this second anti-PrP ligand is not directly connected to a substrate but to a label which reacts with the substrate subsequently added. This surprisingly increases the sensitivity of the assay to a great extent which allows detection and quantification of even very low levels of PrP protein in samples. Therefore, this method is suitable for the detection of low amounts of the PrP antigen and therefore especially suitable to detect also the low level of protein in plasma samples of healthy people.

It is furthermore possible to detect and quantify pathogenic PrP^{res} protein in a sample whereby in a first step PrP^{sen} protein in a sample is eliminated by incubating the sample with proteinase K. The further steps are identical to the above mentioned method for detection and quantification of PrP protein.

A further aspect of the present invention concerns a kit comprising a set of reagents to determine the concentration of PrP protein in a sample, said set of reagents comprising

(a) a solid phase having bound thereto a first anti-PrP ligand;

(b) a first reagent comprising a second labelled anti-PrP ligand;

(c) a second reagent comprising a substrate which reacts with the label of said ligand, the reaction between said substrate and said label resulting in a quantifiable signal; and

(d) optionally a third reagent comprising a control sample comprising a defined concentration of a PrP protein.

A further aspect of the present invention is a kit comprising a set of reagents to determine the concentration of pathogenic PrP^{res} protein in a sample, said set of reagents comprising

(a) a solid phase having bound thereto a first anti-PrP ligand;

(b) a first reagent comprising a second labelled anti-PrP ligand;

(c) a second reagent comprising a substrate which reacts with the label of said ligand, the reaction between said substrate and said label resulting in a quantifiable signal;

(d) a third reagent comprising proteinase K; and

(e) optionally a fourth reagent comprising a control sample comprising a defined concentration of a PrP^{res} protein.

The above mentioned terms and definitions relate not only to the method but also to the above mentioned kit.

### EXAMPLES

For the present examples plasma samples from patients classed as definitely or probably having CJD (30 sporadic and 1 iatrogenic) and from 11 patients with other neurological disorders were collected by the Department of Neurology, University of Göttingen, Germany. Control samples from healthy subjects were obtained either from the University of Göttingen, or from anonymous plasma donations from a commercial plasmapheresis center (Baxter Hyland Immuno, Vienna, Austria). All procedures followed the ethical standards of the committees on human experimentation of the respective institutions.

Plasma samples were stored at −18° C. until subjected to the ELISA assay. All tests were carried out four times and the mean value for each sample was calculated and used for analysis of results.

### Example 1

Development of a Sandwich ELISA for Detection of Human PrP

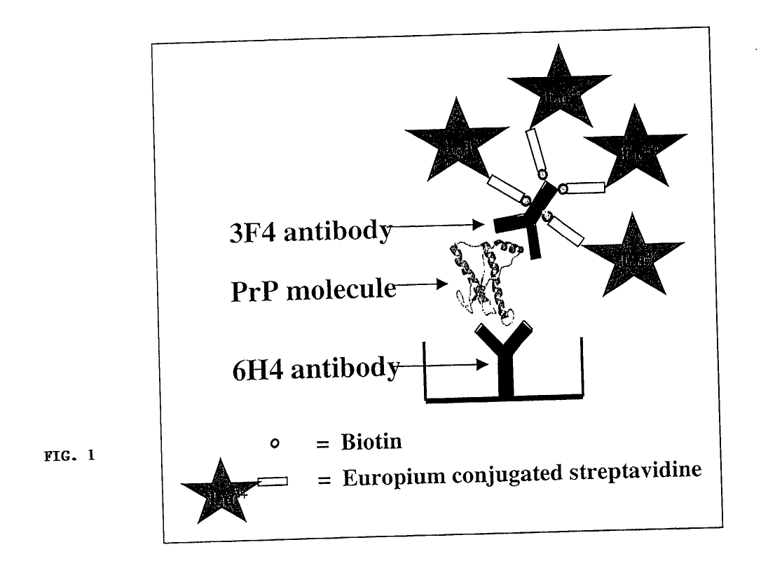

PrP concentrations in plasma samples are measured by a sandwich ELISA using the monoclonal antibody 6H4 (Prionics, Zurich, Switzerland) as the capture ligand and biotinylated monoclonal 3F4 (Senetek, St. Louis, Mo.) as the detection antibody as described in FIG. 1. The monoclonal 3F4 antibody 1 is labelled by biotin 2 and is therefore able to bind several europium-conjugated streptavidin molecules 3, thus enhancing the fluorescence signal. Each well of the microtiter plate is incubated with 150 μl of 6H4 4 that has been diluted 1:1500 in 200 mM carbonate buffer, pH 9.6, for 12 hours at 4° C. The wells are then blocked with 200 μl of 1%-gelatine in phosphate buffered saline (PBS) and incubated for 2 hours at 37° C. Then 150 μl of a solution containing either the sample, the respective concentrations of calibrators, or controls (diluted in PBS containing 0.05% [v/v] Tween 20, PBST) is added and incubated overnight together with 50 μl of the biotin-labelled 3F4 antibody diluted 1:100 in PBST buffer (stock solution, approximately 0.5 μg/ml), thereby allowing PrP molecules 5 to bind to 6H4 antibodies. The 3F4 antibody is then detected with a time-resolved dissociation-enhanced fluoroimmunoassay (DELFIA, EG & G Wallac, Turku, Finland) using 150 μl of streptavidin europium diluted 1:200 with the assay buffer (stock solution, 0.5 μg/ml). Finally, the signal was measured (and expressed in counts per second) with a 1420 Multilabel Counter Victro 2 (Wallac) as recommended by the manufacturer. Recombinant hamster PrP (Prionics) is used for calibration of the assay.

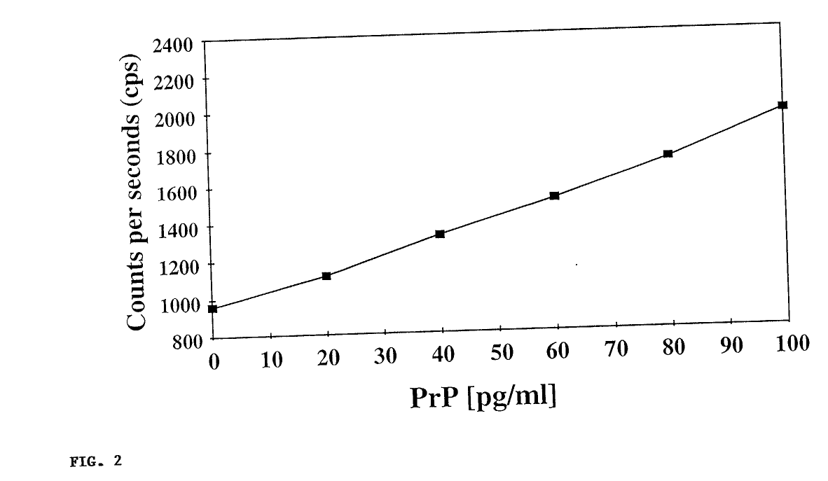

This ELISA technique detects both normal PrP^{sen} and pathogenic PrP^{res} protein because the monoclonal antibodies used (6H4 and 3F4) are not able to distinguish between the PrP isoforms. Using recombinant hamster PrP as a calibrator, a detection limit (in counts per seconds (cps)) of approximately 20 pg/ml in PBS buffer (FIG. 2) and 50 pg/ml in plasma samples pretreated with proteinase K and spiked with recombinant hamster PrP is achieved. Concentration of recombinant hamster PrP containing the 6H4 and the 3F4 epitope sequence is shown in the data points of the curve.

### Example 2

Stability of PrP in Plasma Samples

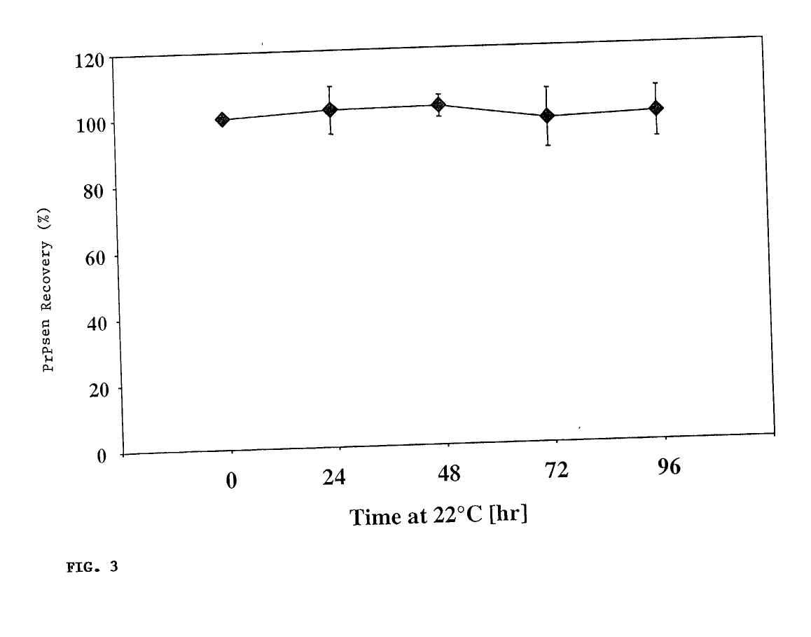

Since the preparation of samples may have differed with regard to storage time, temperature, or centrifugation force, a series of experiments was conducted to investigate the effects of potential variations in these parameters. The designated storage temperature for plasma samples used in the study is −18° C., but the effect of higher temperatures on stability was tested by measuring recovery of PrP in 10 randomly chosen plasma samples: Plasma from 10 representative healthy plasma donors is used for investigation of the stability of PrP^{sen} in plasma over 96 hours at 22° C. The assay is carried out in fresh plasma and in aliquots stored at −18° C. for 24, 28, 72, and 96 hours. The values are calculated in percent of recovery of PrP^{sen} in fresh plasma. The data points show the mean values in 10 plasma samples and the error bars show the standard deviation. Recovery of PrP remaines stable during the entire period (FIG. 3). In addition, no influence of centrifugation force on the level of PrP recovered after centrifugation between 200 and 2500×g is found.

The use of the biotin-conjugated monoclonal antibody 3F4 enabled an increase in the sensitivity of the assay, compared to the method described by MacGregor et al.⁸, who also used two monoclonal antibodies, the second antibody being conjugated directly with europium. Since there is no currently accepted panel or calibrator for ELISA-testing of PrP, a recombinant hamster PrP^{sen} is used for calibration. This method results in a detection limit of 20 pg/ml in buffer solution. Although, due to the high background of other proteins, the assay is slightly less sensitive in plasma (detection limit of 50 pg/ml in samples pretreated with proteinase K and spiked with appropriate amounts of the calibrator), the increased sensitivity makes the ELISA suitable for the detection of low amounts of the PrP^{sen} antigen.

### EXAMPLE 3

Treatment of Plasma Samples with Proteinase K and Spiking Experiments

For elimination of cellular PrP^{sen} in plasma samples by proteinase K treatment, each specimen is diluted 1:5 in PBST buffer and incubated with 50-100 μg proteinase K (Sigma) per ml plasma for 30 minutes at 37° C. The digestion is terminated by addition of 20 mM Pefabloc (Pentapharm, Basel, Switzerland) and heating for 10 minutes at 99° C.

Validation of ELISA is carried out by spiking a representative proteinase K-digested plasma sample with 1% (v/v) recombinant hamster PrP^{sen}, recombinant human PrP^{sen} (Prionics), or pathogenic PrP^{sen} fibrils purified from human brain tissue (kindly provided by H. Budka, University Hospital, Vienna, Austria) obtained by autopsy of the body of a patient with sporadic CJD.

To purify PrP^{res} from the human brain tissue, brain homogenate (10% w/v) is prepared in cold brain lysis buffer (100 mM sodium phosphate, 10 mM EDTA, 0.5% sodium deoxycholate, 0.1 mM phenylmethylsulphonyl fluoride, 0.1 mM N-ethylmaleimide, and 10 mM Tris/HCl, pH 7.4) and centrifuged for 10 minutes at 5000 g. Cellular PrP^{sen} is eliminated from the supernatant by treatment with proteinase K (Sigma, St. Louis, Mo.) at a concentration of 50-100 g/ml for 1 hour at 37° C. Digestion is terminated by addition of 20 mM Pefabloc and heat inactivation for 10 minutes at 100° C. The proteinase K-resistant PrP^{res} fibrils are separated by ultracentrifugation for 2 hours at 170,000 g. The resulting pellet is dissolved in PBS buffer and stirred for 30 minutes at 37° C. The sample is then diluted 1:2 with potassium iodide high salt buffer (100 mM sodium thiosulphate, 36 mM N-lauryl-sarcosine, 10 mM Tris/HCl, and 15% potassium iodide). Finally, an equal volume of 10% potassium iodide high salt buffer is added, overlaid with 20% sucrose and centrifuged again at 180,000 g for 90 minutes. The pellet is then dried, dissolved in 10 μl distilled water, and analyzed by SDS-PAGE and Western blotting.

SDS-PAGE is performed using a Novex 12% homogeneous Tris-glycine gel under reducing conditions and then immunoblotted onto a polyvinylidene difluoride (PVDF) membrane (Novex, San Diego, Calif.). The membranes are blocked with 2% dry milk in 0.05% Tween 20, 150 mM NaCl, and 10 mM Tris-HCl (TBST), pH 8.0, and then incubated with the monoclonal antibody 3F4 diluted 1:5000 in 2% dry milk in TBST buffer at room temperature for 2 hours. After washing, a polyclonal anti-mouse immunoglobulin horseradish peroxidase-linked antibody (Bio-Rad, Richmond, USA, Calif.) diluted 1: 50,000 in TBST is incubated with the membranes for 1 hour at room temperature. The membranes are then washed and developed with the Super Signal chemiluminescent substrate kit (perce, Rockford, Ill.) as recommended by the manufacturer.

Type of PrP Detected by ELISA in Plasma

PrP^{sen} is measured in plasma from a healthy donor (a). The same sample is diluted 1:5 in PBST buffer and treated with proteinase K (50μg/ml) for 30 min at 37° C. (b). The proteinase K digested plasma sample is then subdivided and used for spiking experiments with recombinant hamster PrP (c), recombinant human PrP (d) and pathogenic PrP^{res} fibrils purified from human brain homogenate from a patient with sCJD (e).

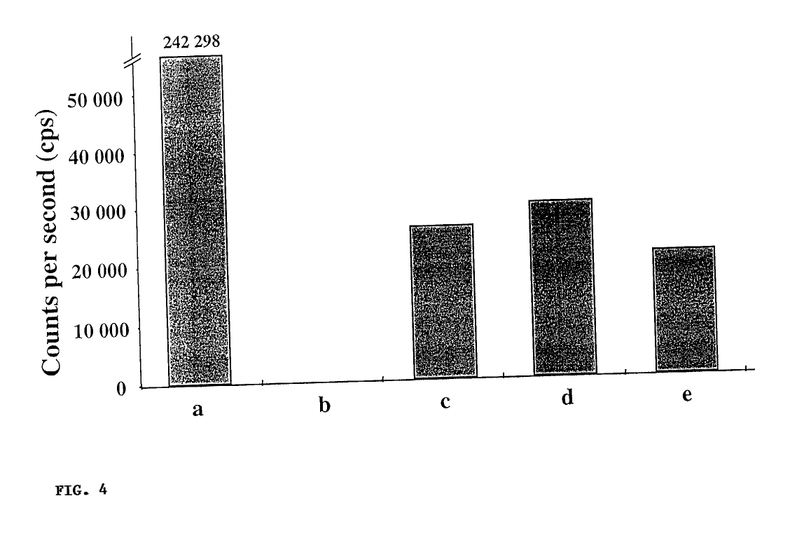

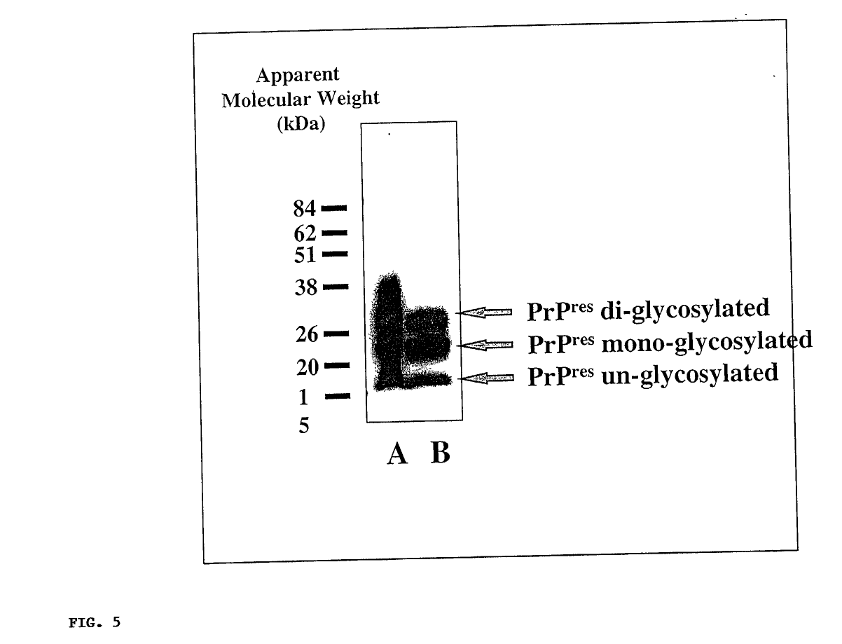

As shown in FIG. 4, ELISA signals are obtained in plasma from normal healthy blood donors before (a), but not after addition of proteinase K (b). Signals are also obtained after spiking proteinase K-digested plasma samples with recombinant hamster PrP^{sen} (c), recombinant human PrP^{sen} (d), and pathogenic PrP^{res} from the brain of a patient with CJD (e). The identity of the pathogenic PrP^{res} used for spiking is shown by Western blot analysis before and after proteinase K treatment in FIG. 5 (the numbers in the left lane indicate the apparent molecular weight in kDa). Brain homogenate from a patient affected with sCJD is used for the preparation of pathogenic PrP^{res} fibrils. After the purification procedure the resulting PrP protein is analyzed by SDS gel electrophoresis and Western blot analysis before (lane A) and after treatment with proteinase K for 1 hour at 37° C. (lane B). Thus, the ELISA assay detects human soluble PrP^{sen} of both recombinant and cellular origin as well as pathogenic PrP^{res}. Due to epitope differences, it does not detect recombinant bovine PrP.

The PrP signal is eliminated by treatment with proteinase K in all samples tested in this study, indicating that the detected protein is PrP^{sen}, not PrP^{res}. If any PrP^{res} is present in plasma samples from patients with sCJD, the amount is not large enough to be detected.

### Example 4

PrP Levels in Healthy Plasma Donors, Patients with CJD, and Patients with other Neurological Diseases

PrP^{sen} levels are examined in plasma from a group of 200 healthy plasma donors aged between 18 and 64 years (median 30 years), from the 31 CJD cases, and from 11 patients with other neurological diseases (Table 1).

TABLE 1
Median plasma concentration of PrP from patients with
CJD and other diseases

| | No. of | Median | |
|---|---|---|---|
| Diagnosis | patients | (ng/mL) | (Range) |
| Creutzfeldt-Jakob disease | | | |
| Definitive sCJD | 16 | 16.0 | (6.2-26.1) |
| Probably sCJD | 14 | 13.5 | (7.4-23.8) |
| Probably iatrogenic CJD | 1 | 10.3 | |
| All CJD | 31 | 14.7 | (6.2-26.1) |
| Other neurological diseases | | | |
| Alzheimer disease | 2 | 18.7 | (17.7-19.7) |
| Depression by dementia of | 1 | 13 | |
| Alzheimer | | | |
| Parkinson disease | 1 | 29.7 | |
| Unclear dementia | 1 | 203 | |
| Other dementia | 1 | 28.6 | |
| Inflammatory brain damage | 2 | 19 | (18.4-21) |
| Other brain damage | 1 | 30.8 | |
| Alcoholism | 1 | 16.8 | |
| State of confusion | 1 | 15.4 | |
| Healthy controls | 200 | 6.2 | (2.5-9.2) |

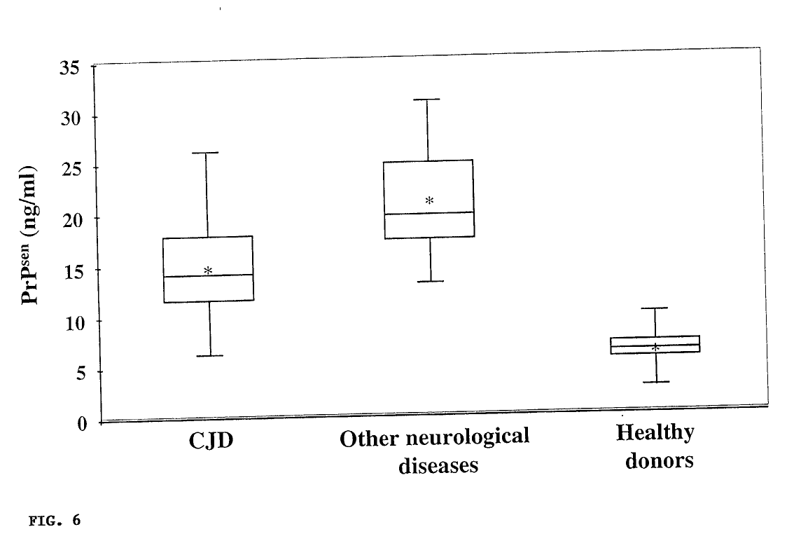

For each group, the median and mean concentration of PrP^{sen}, the 25^{th} and 75^{th} percentiles and the minimum and maximum of the PrP^{sen} are calculated (FIG. 6). The PrP^{sen} concentrations are depicted by box whisker plot. The horizontal lines in each the box represent the median concentration of PrP^{sen}, the asterisk represents the mean, the upper and lower edges of the box represent the 75th and 25^{th} percentile, respectively, and the lines extending below and above the box represent the minimum and maximum values. In healthy plasma donors (box 3), the median plasma concentration of PrP^{sen} is 6.2 ng/ml and the mean is 6.1 ng/ml (this value could be the threshold value for this example) (minimum, 2.5 ng/ml; 25^{th} percentile, 2.5 ng/ml; 75^{th} percentile, 7.0 ng/ml; maximum, 9.2 ng/ml). The median plasma concentration of PrP^{sen} in the CJD group (box 1) (both definite and probable cases) was 14.2 ng/ml and the mean is 14.7 ng/ml (minimum 6.2 ng/ml; 25^{th} percentile, 11.6 ng/ml; 75^{th} percentile, 17.8 ng/ml; maximum, 26.1 ng/ml). In the patients with other neurological diseases (box 2), the median plasma concentration of PrP^{sen} is 17.3 ng/ml and the mean is 21.0 ng/ml (minimum, 13.0 ng/ml; 25^{th} percentile, 17.3 ng/ml; 75^{th} percentile, 24.8 ng/ml; maximum, 30.8 ng/ml).

In 27 of the 31 CJD patients (87%), and in all patients with other neurodegenerative diseases, the PrP^{sen} level is higher than that of the highest value in the control group of healthy plasma donors. In conclusion, this data indicates that an increased level of PrP in plasma samples is a surrogate marker for the presence of neurological disease, including CJD. The ELISA described above is an important tool in the rapid screening of plasma samples for various purposes; in clinical use, in screening plasma donations in order to increase the safety of blood products, and in the study of the course of diseases such as sCJD and vCJD. There has been discussion regarding possible correlations between CJD and vCJD and changes in levels of proteins such as S-100, NSE or 14-3-3 proteins,¹⁸ which are nonspecific surrogate markers for brain damage. Although Otto et al.¹⁶ showed that S-100 protein might serve as a surrogate plasma marker for CJD, the low levels of the specific protein found in plasma and its cross-reactivity to various isoenzymes⁹ make it difficult to use in a routine test. The use of NSE in plasma has similar disadvantages.¹¹ The determination of 14-3-3 proteins seems to be the preferred diagnostic test for CJD.^{15,18,19} However, 14-3-3 proteins can be analyzed only in cerebrospinal fluid, which is not acceptable for routine screening. In contrast to these proteins, PrP is found in relatively high concentrations in plasma and can be measured even by a simple sandwich ELISA, however, the above described sensitive ELISA is preferred in order to be able to detect even very low PrP concentrations in samples.

### Example 5

PrP Level and Age

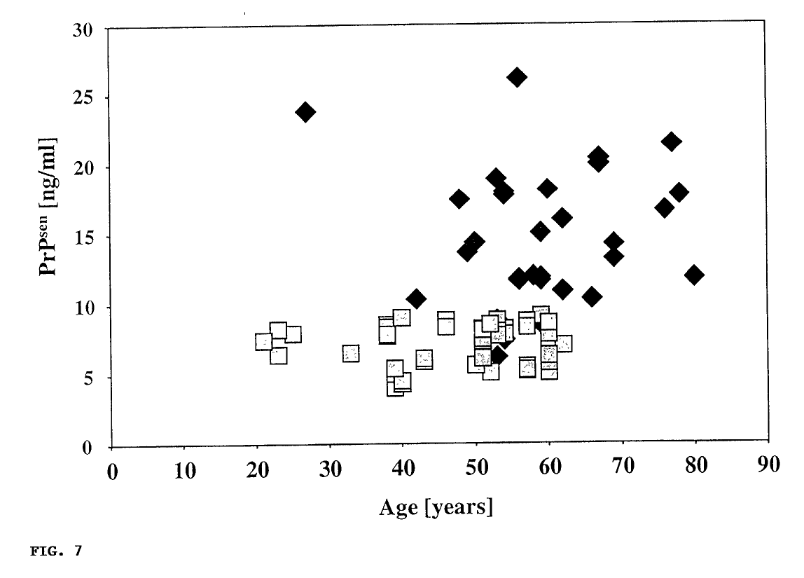

It was conceivable that the patient groups had higher PrP levels because they were older than the healthy blood donors. Therefore, the relationship between PrP^{sen} and age was examined, but no correlations were found. The PrP^{sen} concentration of plasma samples is shown from a group of 50 healthy plasma donors between 21 and 61 years (□) and the PrP^{sen} concentration from 31 CJD cases between 27 and 80 years (♦) (FIG. 7). No correlation was found between age and PrP level within any of these groups; therefore, the high value of PrP protein in patients with neurological disorders is due to their illness and not to their age.

To summarize, the ELISA described above can be used for the screening of plasma samples for pathogenic PrP^{sen} and recombinant human PrP^{sen}, but also pathogenic PrP^{res} purified from human brain. Although the assay does not differentiate between the normal PrP^{sen} and the pathogenic PrP^{res} protein because the monoclonal antibodies (6H4 and 3F4) used do not distinguish between the individual PrP isoforms, treatment of a sample with proteinase K to digest the PrP^{sen} allows measurement of PrP^{res}. However, the amount of PrP^{res} in plasma from patients with sCJD would not be expected to be large enough to be detectable, even at the level of sensitivity achieved by the assay. Indeed, PrP^{res} was not detected in any of the plasma samples from patients with sCJD.

All publications and patent documents cited in this application are incorporated by reference in their entirety for all purposes to the same extent as if each individual publication or patent document were so individually denoted.

References

1. Stahl N, Borchelt D R, Hsiao K, et al. Scrapie prion protein contains a phosphatidylinositol glycolipid. Cell 1987; 51:229-40.

2. Kretzschmar H A, Prusiner S B, Stowring L E, et al. Scrapie prion proteins are synthesized in neurons. Am. J. Pathol. 122 (1986) 1-5.

3. Moser M, Colello R J, Pott U, et al. Developmental expression of the prion protein gene in glial cells. Neuron 1995; 509-17.

4. Brandner S, Klein M A, Aguzzi A. A crucial role for B cells in neuroinvasive scrapie. Transfus Clin Biol 1999; 1:17-3.

5. Brown P. B lymphocytes and neuroinvasion. Nature 1997; 390:662-3.

6. Barclay G R, Hope J, Birkett C R, et al. Distribution of cell-associated prion protein in normal adult blood determined by flow cytometry. Br J Haematol 1999; 107:804-14.

7. Holada K, Mondoro T H, Muller J, et al. Increased expression of phosphatidylinositol-specific phospholipase C resistant prion proteins on the surface of activated platelets. Br J Haematology 1998; 103:276-2.

8. MacGregor I, Hope J, Barnard G, et al. Application of a time-resolved fluoroimmunoassay for the analysis of normal prion protein in human blood and its components. Vox Sang 1999; 77:88-6.

9. Heizmann C W. Diagnosis of Creutzfeldt-Jakob disease. Subtypes of S100 proteins must be identified. BMJ 1999; 318:538-41.

10. Otto M, Wilffang J, Tumani H, et al. Elevated levels of tau-protein in cerebrospinal fluid of patients with Creutzfeldt-Jakob disease. Neurosci Lett 1997; 225:210-2.

11. Beaudry P, Cohen P, Brandel J P, et al. 14-3-3 protein, neuron-specific enolase, and S-100 protein in cerebrospinal fluid of patients with Creutzfeldt-Jakob disease. Dement Geriatr Cogn Disord 1999; 10:40-6.

12. Zerr I, Bodemer M, Otto M, et al. Diagnosis of Creutzfeldt-Jakob disease by two-dimensional gel electrophoresis of cerebrospinal fluid. Lancet 1996; 348:846-9.

13. Jimi T, Wakayama Y, Shibuya 5, et al. High levels of nervous system-specific proteins in cerebrospinal fluid in patients with early stage Creutzfeldt-Jakob disease. Clin Chim Acta 1992; 11:37-6.

14. Adams D H. The “infective” process in scrapie and human spongiform encephalopathy disease. Med Hypotheses 1995; 145:214-8.

15. Zerr I, Bodemer M, Gefeller 0, et al. Detection of 14-3-3 protein in the cerebrospinal fluid supports the diagnosis of Creutzfeldt-Jakob disease. Ann Neurol 1998; 43:32-0.

16. Otto M, Wiltfang J, Schutz E, et al. Diagnosis of Creutzfeldt-Jakob disease by measurement of S100 protein in serum: prospective case-control study. BMJ 1998; 316:577-2.

17. Hemmilä I, Dakubu 5, Mukkala V-M, et al. Europium as a label in time-resolved immunofluorometric assays. Anal Biochem 1984; 317:738-9.

18. Wiltfang J, Otto M, Baxter H C, et al. Isoform pattern of 14-3-3 proteins in the cerebrospinal fluid of patients with Creutzfeldt-Jakob disease. J Neurochem 1999; 73:2485-0.

19. Saiz A, Graus F, Dalmau J, et al. Detection of 14-3-3 brain protein in the cerebrospinal fluid of patients with paraneoplastic neurological disorders. Ann Neurol 1999; 46:774-7.

What is claimed is:

1. A method for the detection of neurological disorders in a patient comprising
(a) measuring the concentration of PrP protein in a biological fluid sample of said patient; and
(b) determining whether said concentration of said PrP protein is above or below a predetermined threshold value, whereby the concentration above said predetermined threshold value identifies a patient with a neurological disorder.

2. The method according to claim 1 wherein said measuring of said PrP protein is carried out immunologically.

3. The method according to claim 2 wherein said measuring comprises
(a) incubating said plasma sample with a labelled anti-PrP ligand;
(b) determining the amount of bound labelled ligand.

4. The method according to claim 3 wherein said incubating comprises
(a) immobilizing said PrP protein which is present in said plasma sample to a solid phase; and
(b) adding to said bound PrP protein said labelled anti-PrP ligand.

5. The method according to claim 4 wherein said immobilizing said PrP protein comprises
(a) immobilizing a first anti-PrP ligand to said solid phase;
(b) adding said plasma sample so that said PrP protein binds to said first anti-PrP ligand and is immobilized to said solid phase.

6. The method according to claim 3 said determining said amount of bound labelled ligand comprising
(a) adding a substrate which reacts with the label of said ligand;
(b) the reaction between said substrate and said label resulting in a quantifiable signal; and
(c) measuring said signal.

7. The method according to claim 6 said determining said amount of label of bound ligand comprising comparing said signal of said plasma sample with a measured signal of a control sample comprising a defined concentration of PrP protein.

8. The method according to claim 6 wherein the quantifiable signal is a quantifiable optical signal.

9. The method according to claim 8 wherein the reaction between said substrate and the label of said ligand results in a fluorescent signal.

10. The method according to claim 9 wherein said substrate is selected from the group consisting of europium, terbium, gadolinium, samarium and dysprosium.

11. The method according to claim 10 wherein said substrate has bound thereto avidin or streptavidin.

12. The method according to claim 11 wherein said labelled ligand is biotinylated.

13. A method for the detection of neurological disorders in a patient, said neurological disorders being selected from the group consisting of Creutzfeldt-Jakob disease (CJD), Alzheimer disease, depression by dementia of Alzheimer, Parkinson disease, dementia, inflammatory brain damage, Alcoholism, or state of confusion, said detection comprising
(a) measuring the concentration of PrP protein in a plasma sample of said patient; and
(b) determining whether said concentration of said PrP protein is above or below a predetermined threshold value, whereby the concentration above said predetermined threshold value identifies a patient with a neurological disorder.

14. The method according to claim 13 wherein said measuring of said PrP protein is carried out immunologically.

15. The method according to claim 14 wherein said measuring comprises
(a) incubating said plasma sample with a labelled anti-PrP ligand;
(b) determining the amount of bound labelled ligand.

16. The method according to claim 15 wherein said incubating comprises
(a) immobilizing said PrP protein which is present in said plasma sample to a solid phase; and
(b) adding to said bound PrP protein said labelled anti-PrP ligand.

17. The method according to claim 16 wherein said immobilizing said PrP protein comprises
(a) immobilizing a first anti-PrP ligand to said solid phase;
(b) adding said plasma sample so that said PrP protein binds to said first anti-PrP ligand and is immobilized to said solid phase.

18. The method according to claim 15 said determining said amount of label of bound ligand comprising
(a) adding a substrate which reacts with the label of said bound ligand;
(b) the reaction between said substrate and said label resulting in a quantifiable signal; and
(c) measuring said signal.

19. The method according to claim 18 said determining said amount of label of bound ligand comprising comparing said signal of said biolocial fluid sample with a measured signal of a control sample comprising a defined concentration of PrP protein.

20. The method according to claim 18 wherein the quantifiable signal is a quantifiable optical signal.

21. The method according to claim 20 wherein the reaction between said substrate and the label of said ligand results in a fluorescent signal.

22. The method according to claim 21 wherein said substrate is selected from the group consisting of europium, terbium, gadolinium, samarium and dysprosium.

23. The method according to claim 22 wherein said substrate has bound thereto avidin or streptavidin.

24. The method according to claim 23 wherein said labelled ligand is biotinylated.

25. A method for the detection and quantification of PrP protein in a sample comprising
(a) immobilizing a first anti-PrP ligand to a solid phase;
(b) incubating said sample with said first anti-PrP ligand so that said PrP protein binds to said first ligand;
(c) adding a second labelled anti-PrP ligand so that said second ligand binds to said PrP protein;
(d) adding a substrate which reacts with the label of said second bound ligand;
(e) the reaction between said substrate and said label resulting in a quantifiable signal;
(f) measuring said signal; and
(g) determining the concentration of PrP protein in said sample, preferably by comparing said signal of said sample with a signal of a control sample comprising a defined concentration of PrP protein.

26. The method according to claim 25 wherein the quantifiable signal is a quantifiable optical signal.

27. The method according to claim 26 wherein the reaction between said substrate and said label results in a fluorescent signal.

28. The method according to claim 27 wherein said substrate is selected from the group consisting of europium, terbium, gadolinium, samarium and dysprosium.

29. The method according to claim 28 wherein said substrate has bound thereto avidin or streptavidin and said labelled ligand is biotinylated.

30. A method for the detection and quantification of pathogenic PrP^{res} protein in a sample comprising
(a) elimination of PrP^{sen} protein in said sample by incubating said sample with proteinase K;
(b) immobilizing a first anti-PrP ligand to a solid phase;
(c) incubating said proteinase K digested sample with said first anti-PrP ligand so that non-digested PrP^{res} protein binds to said first ligand;
(d) adding a second labelled anti-PrP ligand so that said second ligand binds to said PrP^{res} protein;
(e) adding a substrate which reacts with the label of said second bound ligand;
(f) the reaction between said substrate and said label resulting in a quantifiable signal;
(g) measuring said signal; and
(h) determining the concentration of PrP protein in said sample, preferably by comparing said signal of said sample with a signal of a control sample comprising a specific concentration of PrP^{res} protein.

31. The method according to claim 30 wherein the quantifiable signal is a quantifiable optical signal.

32. The method according to claim 31 wherein the reaction between said substrate and said label results in a fluorescent signal.

33. The method according to claim 32 wherein said substrate is selected from the group consisting of europium, terbium, gadolinium, samarium and dysprosium.

34. The method according to claim 33 wherein said substrate has bound thereto avidin or streptavidin.

35. The method according to claim 34 wherein and said labelled ligand is biotinylated.

36. A kit comprising a set of reagents to determine the concentration of PrP protein in a sample, said set of reagents comprising
(a) a solid phase having bound thereto a first anti-PrP ligand;
(b) a first reagent comprising a second labelled anti-PrP ligand;
(c) a second reagent comprising a substrate which reacts with the label of said second ligand, the reaction between said substrate and said label resulting in a quantifiable signal.

37. The kit according to claim 36 further comprising a third reagent comprising a control sample with a defined concentration of a PrP protein.

38. The kit according to claim 37 wherein said quantifiable signal is a quantifiable optical signal.

39. The kit according to claim 38 wherein said substrate is selected from the group consisting of europium, terbium, gadolinium, samarium and dysprosium.

40. The kit according to claim 39 wherein said substrate has bound thereto avidin or streptavidin.

41. The kit according to claim 40 wherein said labelled ligand is biotinylated.

42. The kit according to claim 36 for the detection of neurological disorders in a patient wherein said sample is a biological fluid sample of said patient.

43. The kit according to claim 42 wherein said neurological disorders are selected from the group consisting of Creutzfeldt-Jakob disease (CJD), Alzheimer disease, depression by dementia of Alzheimer, Parkinson disease, dementia, inflammatory brain damage, Alcoholism, or state of confusion.

44. A kit comprising a set of reagents to determine the concentration of pathogenic PrP^{res} protein in a sample, said set of reagents comprising
(a) a solid phase having bound thereto a first anti-PrP ligand;
(b) a first reagent comprising a second labelled anti-PrP ligand;
(c) a second reagent comprising a substrate which reacts with the label of said ligand, the reaction between said substrate and said label resulting in a quantifiable signal;
(d) a third reagent comprising proteinase K.

45. The kit according to claim 44 further comprising a fourth reagent comprising a control sample with a defined concentration of a PrP^{res} protein.

46. The kit according to claim 45 wherein said quantifiable signal is a quantifiable optical signal.

47. The kit according to claim 46 wherein said substrate is selected from the group consisting of europium, terbium, gadolinium, samarium and dysprosium.

48. The kit according to claim 47 wherein said substrate has bound thereto avidin or streptavidin.

49. The kit according to claim 48 wherein said labelled ligand is biotinylated.
